(19) United States
(12) Patent Application Publication
(10) Pub. No.: US 2025/0369892 A1
(43) Pub. Date: Dec. 4, 2025

(54) DEVICE, SYSTEM AND METHOD FOR DETECTING SUBSTANCES IN LIQUID TO BE TESTED USING PLASMA SPECTROSCOPY

(71) Applicant: NATIONAL TAIWAN UNIVERSITY, Taipei (TW)

(72) Inventors: CHENG-CHE HSU, Taipei City (TW); CHING-YUAN WANG, Taipei City (TW); JIAN-TI CHEN, Taipei City (TW)

(73) Assignee: NATIONAL TAIWAN UNIVERSITY, Taipei (TW)

(21) Appl. No.: 18/934,260

(22) Filed: Nov. 1, 2024

(30) Foreign Application Priority Data
Jun. 3, 2024 (TW) 113120496

(51) Int. Cl. G01N 21/69; G01N 33/18

(52) U.S. Cl. CPC G01N 21/69; G01N 33/1813

(57) ABSTRACT

The present disclosure discloses a device for detecting substances in a liquid to be tested using plasma spectroscopy, including an electrode, at least a portion of which is configured to be disposed in the liquid to be tested and is suitable to contact the liquid to be tested, the electrode being suitable to generate a plasma in the liquid to be tested by applying an external voltage, wherein the plasma is located in a bubble generated by the external voltage; a light detector, configured to detect the emission spectrum generated by the plasma in the bubble, wherein the light detector is an optical fiber, and there is no focusing element between the light detector and the plasma.

### CROSS-REFERENCE TO RELATED APPLICATION

This non-provisional application claims priority under 35 U.S.C. § 119 (a) on patent application No. 113120496 filed in Taiwan, R.O.C. on Jun. 3, 2024, the entire contents of which are hereby incorporated by reference.

### BACKGROUND OF THE INVENTION

### 1. Field of the Invention

The present disclosure relates to a device, system and method of testing a substance in a liquid, and in particular to a device, system and method of testing a substance in a liquid under test through plasma optical emission spectrum.

### 2. Description of the Related Art

Water quality tests are applicable to all areas of water, such as natural waterbodies, household water, industrial water, discharged wastewater, and water being used in an industrial manufacturing process, as well as scenes and sites where environmental monitoring, hygiene monitoring, industrial safety monitoring, and production process monitoring take place, to test the water quality in all areas of water and thereby determine whether related environmental safety condition, production process condition or industrial wastewater drainage meets environmental protection standards. Water quality tests are commonly conducted to analyze the types and contents of heavy metals.

It is necessary to meet continuous monitoring needs in various sites. Take industrial sites as an example, real-time production-line heavy metal tests are of vital importance from an industrial perspective. However, to the detriment of its industrial use, conventional industrial heavy metal production-line continuous test technology has drawbacks as follows: high device cost, exclusive use of one single apparatus in testing one single metal only, poor tolerance because of high susceptibility to interference from other substances, discharging other toxic waste liquid in the course of the test. Furthermore, conventional industrial heavy metal production-line continuous test technology requires apparatuses that are bulky, too excessively intricate to achieve ease of use, expensive, and not portable to carry to various sites to monitor substances in an aqueous solution.

For instance, experiment-level techniques of testing heavy metals include Inductively Coupled Plasma Optical Emission Spectroscopy (ICP-OES), and Flame Atomic Absorption Spectroscopy (FAAS), both being applied to various heavy metal tests and featuring a low concentration detection limit. However, they require expensive instruments, complicated sample preprocessing steps, and lengthy technician training courses, not to mention that they are unable to yield test results efficiently and quickly. On the other hands, commercially-available, portable heavy metal test kits, such as chromogenic reactions, and anodic stripping voltammetry, are portable, quick to test, and easy to use. However, the commercially-available, portable heavy metal test kits have a drawback: interactions between metals are likely to occur to the detriment of test signals.

Therefore, none of the existing techniques or devices of testing a substance in an aqueous solution meets all advantageous requirements as follows: portable, compact, easy to use, incurring low cost, capable of testing multiple heavy metals, quick to test, and insusceptible to mutual interference between different metals.

### BRIEF SUMMARY OF THE INVENTION

To enable a substance in an aqueous solution to be tested efficiently, quickly and accurately, it is feasible to employ an aqueous solution plasma technique that entails producing a plasma in an aqueous solution with an electrode, testing an optical emission spectrum of the plasma optically, and analyzing the optical emission spectrum of the plasma to identify substances contained in the aqueous solution.

However, existing aqueous solution plasma techniques are confronted with the difficulties in effectively collecting light signals emitted from a plasma. For example, optical emission spectrum signals generated from a plasma are susceptible to interference from bubbles generated from a plasma, greatly reducing the intensity and accuracy of the plasma optical emission spectrum signals collected. Unequal sizes of bubbles, movements and changes of bubbles, generation and destruction of bubbles, and optical phenomena, such as reflection and refraction caused by gas-liquid interfaces of bubbles, greatly affect the intensity and accuracy of the plasma optical emission spectrum signals collected. In addition, light signals generated from a plasma are likely to be affected by an aqueous solution per se, greatly reducing the intensity and accuracy of the plasma optical emission spectrum signals collected.

Therefore, it is an objective of the disclosure to provide a device, system and method of testing a substance in an aqueous solution, with the device being portable, compact, easy to use, incurring low cost, capable of testing multiple heavy metals, quick to test, and insusceptible to mutual interference between different metals, in order for the device, system and method to surpass existing aqueous solution plasma techniques in insusceptibility to characteristics of bubbles and aqueous solutions, the intensity and accuracy of the plasma optical emission spectrum signals, as well as testing the types of substances in an aqueous solution quickly, effectively and accurately.

Therefore, the disclosure provides a device, system and method of testing a substance in a liquid under test through plasma optical emission spectrum to greatly enhance the intensity and accuracy of the plasma optical emission spectrum signals in aqueous solution plasma techniques.

An aspect of the disclosure provides a device of testing a substance in a liquid under test through plasma optical emission spectrum, comprising: an electrode having at least a portion adapted to be disposed inside the liquid under test and adapted to be in contact with the liquid under test, wherein the electrode is adapted to produce a plasma in the liquid under test under an applied voltage, and the plasma is located in a bubble generated under the applied voltage; and a light detection element adapted to detect an optical emission spectrum generated from the plasma in the bubble, the light detection element being an optical fiber, wherein no light-focusing component is present between the light detection element and the plasma.

Regarding the device of testing a substance in a liquid under test through plasma optical emission spectrum, wherein the light detection element has at least a portion adapted to be disposed inside the liquid under test and comprising a light-receiving end adapted to be located in the bubble to detect the optical emission spectrum generated from the plasma in the bubble.

Regarding the device of testing a substance in a liquid under test through plasma optical emission spectrum, wherein the light detection element is oriented at a first angle to a normal of a contact surface between the electrode and the liquid under test, with an included angle of 0° defined between the first angle and the normal of the contact surface.

Regarding the device of testing a substance in a liquid under test through plasma optical emission spectrum, wherein the light detection element has at least a portion adapted to be disposed inside the liquid under test and comprising a light-receiving end separated from the electrode by a distance configured to allow the light-receiving end to be adapted to be located within scope of the bubble.

Regarding the device of testing a substance in a liquid under test through plasma optical emission spectrum, wherein at least a portion of the light detection element comprises a light-receiving end separated from the electrode by a distance ranging from 0.1 mm to 4 mm.

Regarding the device of testing a substance in a liquid under test through plasma optical emission spectrum, wherein the light detection element is oriented at a second angle to a normal of a contact surface between the electrode and the liquid under test, with an included angle of 90° defined between the second angle and the normal of the contact surface.

Regarding the device of testing a substance in a liquid under test through plasma optical emission spectrum, wherein at least a portion of the light detection element is adapted to be disposed inside the liquid under test and comprises a light-receiving end separated from the electrode by a distance configured to allow the light-receiving end to be adapted to be located within scope of the bubble.

Regarding the device of testing a substance in a liquid under test through plasma optical emission spectrum, wherein at least a portion of the light detection element comprises a light-receiving end separated from the electrode by a distance ranging from 0.05 mm to 3.5 mm.

Regarding the device of testing a substance in a liquid under test through plasma optical emission spectrum, wherein the light detection element is oriented at a third angle to a normal of a contact surface between the electrode and the liquid under test, allowing an included angle defined between the third angle and the normal of the contact surface to fall between a first angle and a second angle, with an included angle of 0° defined between the first angle and the normal of the contact surface, with an included angle of 90° defined between the second angle and the normal of the contact surface.

Regarding the device of testing a substance in a liquid under test through plasma optical emission spectrum, wherein the light detection element has at least a portion adapted to be disposed inside the liquid under test and comprising a light-receiving end separated from the electrode by a distance configured to allow the light-receiving end to be adapted to be located within scope of the bubble.

Regarding the device of testing a substance in a liquid under test through plasma optical emission spectrum, wherein at least a portion of the light detection element comprises a light-receiving end separated from the electrode by a distance ranging from 0.05 mm to 3.5 mm.

Regarding the device of testing a substance in a liquid under test through plasma optical emission spectrum, wherein at least a portion of the light detection element is adapted to be disposed inside the liquid under test and comprises a light-receiving end separated from the electrode by a distance ranging from 0.05 mm to 10 mm.

An aspect of the disclosure provides a system of testing a substance in a liquid under test through plasma optical emission spectrum, comprising: the device of testing a substance in a liquid under test through plasma optical emission spectrum; a sample chamber configured to retain the electrode and the light detection element and receive the liquid under test; a spectrometer coupled to the light detection element and configured to analyze an optical emission spectrum generated from a plasma in the bubble and detected by the light detection element; and a power coupled to the electrode and configured to provide the applied voltage to the electrode.

The system of testing a substance in a liquid under test through plasma optical emission spectrum, further comprising an electronic device electrically connected to the spectrometer and configured to analyze the optical emission spectrum through the spectrometer.

Regarding the system of testing a substance in a liquid under test through plasma optical emission spectrum, wherein the electronic device is configured to be signal-connected to an external device to provide an analysis result about the optical emission spectrum of the liquid under test to the external device in real time. The system of testing a substance in a liquid under test through plasma optical emission spectrum, further comprising an electronic device electrically connected to the power and configured to set a parameter of the applied voltage through the power to adjust the plasma produced in the liquid under test.

The system of testing a substance in a liquid under test through plasma optical emission spectrum, further comprising an electronic device electrically connected to the power and the spectrometer and configured to synchronize the power and the spectrometer so as to synchronize production of the plasma and reception of the optical emission spectrum.

An aspect of the disclosure provides a method of testing a substance in a liquid under test through plasma optical emission spectrum, comprising the steps of: providing an electrode in a liquid under test; allowing the electrode to come into contact with the liquid under test; applying an applied voltage to produce a plasma in the liquid under test; and detecting, by a light detection element, an optical emission spectrum generated from the plasma, wherein the plasma is located in a bubble generated under the applied voltage, wherein the light detection element is an optical fiber, and no any light-focusing component is present between the light detection element and the plasma.

Regarding the method of testing a substance in a liquid under test through plasma optical emission spectrum, wherein the step of detecting, by a light detection element, an optical emission spectrum generated from the plasma further comprises the steps of: placing at least a portion of the light detection element in the liquid under test; placing a light-receiving end included in at least a portion of the light detection element in a bubble; and detecting directly an optical emission spectrum generated from the plasma in the bubble.

Therefore, the disclosure provides a device, system and method of testing a substance in a liquid under test through plasma optical emission spectrum, using an optical fiber not having any light-focusing component as a light detection element to effectively prevent plasma optical emission spectrum from undergoing signal attenuation and interference otherwise arising from marked interference-induced light receiving focus alteration because of unequal sizes of bubbles, changes and movements of bubbles, generation and destruction of bubbles, attachment of bubbles to the light detection element, and optical phenomena, such as reflection and refraction caused by gas-liquid interfaces of bubbles, when the light signals are focused toward the optical fiber with optical components, such as lenses. Owing to the optical fiber not having any light-focusing component, the light signals of the optical emission spectrum generated by plasma upon the generation of the plasma and bubbles in the liquid under test under an applied voltage are effectively and completely collected to obtain effective and precise results of analyses and tests of various substances in the liquid under test. The simple arrangement of the electrode and the light detection element is conducive to performing the tests through plasma optical emission spectrum, achieving advantages, such as portability, compactness, ease of use, and low cost, testing multiple heavy metals simultaneously, performing tests quickly, and rare mutual interference between different metals.

### BRIEF DESCRIPTION OF THE DRAWINGS

FIG. 1A (PRIOR ART) is a schematic view of a conventional plasma testing device that receives light through a lens assembly outside a solution.

FIG. 1B (PRIOR ART) is a schematic view of a conventional plasma testing device that receives light through a lens assembly inside a solution.

FIG. 1C (PRIOR ART) is a schematic view of the trend of the intensity of signals obtained by a conventional plasma testing device that receives light through a lens assembly inside a solution.

FIG. 2A is a schematic view of the device of testing a substance in a liquid under test through plasma optical emission spectrum according to an embodiment of the disclosure.

FIG. 2B is a schematic view of the device of testing a substance in a liquid under test through plasma optical emission spectrum according to an embodiment of the disclosure.

FIG. 3 is a schematic view of comparing the intensity of signals obtained according to the prior art with the intensity of signals obtained by the device of testing a substance in a liquid under test through plasma optical emission spectrum according to an embodiment of the disclosure.

FIG. 4 is a schematic view of comparing the spectrum distribution obtained according to the prior art with the spectrum distribution obtained by the device of testing a substance in a liquid under test through plasma optical emission spectrum according to an embodiment of the disclosure.

FIG. 5A is a schematic view of the device of testing a substance in a liquid under test through plasma optical emission spectrum according to an embodiment of the disclosure.

FIG. 5B is a schematic view of the device of testing a substance in a liquid under test through plasma optical emission spectrum according to an embodiment of the disclosure.

FIG. 6A is a schematic view of comparing the spectrum intensity obtained as a result of different distances between a light detection element and an electrode according to an embodiment of the disclosure.

FIG. 6B is a schematic view of comparing the spectrum intensity obtained as a result of different distances between the light detection element and the electrode according to an embodiment of the disclosure.

FIG. 7A is a schematic view of different angles between the light detection element and the electrode according to an embodiment of the disclosure.

FIG. 7B is a schematic view of comparing the spectrum intensity obtained as a result of different angles between the light detection element and the electrode according to an embodiment of the disclosure.

FIG. 7C is a schematic view of comparing the spectrum intensity obtained as a result of different angles between the light detection element and the electrode according to an embodiment of the disclosure.

FIG. 8 are schematic views of the spectrum intensity obtained by applying different applied voltages to the electrode according to an embodiment of the disclosure.

FIG. 9 are schematic views of the spectrum intensity obtained by using different electrode sizes according to an embodiment of the disclosure.

FIG. 10 are schematic views of the spectrum intensity obtained by applying different pulse times to the electrode according to an embodiment of the disclosure.

FIG. 11 is a schematic view of the system of testing a substance in a liquid under test through plasma optical emission spectrum according to an embodiment of the disclosure.

FIG. 12 is a schematic view of the system of testing a substance in a liquid under test through plasma optical emission spectrum according to an embodiment of the disclosure.

FIG. 13 is a schematic view of the system of testing a substance in a liquid under test through plasma optical emission spectrum according to an embodiment of the disclosure.

FIG. 14 is a schematic view of a process flow of a method of testing a substance in a liquid under test through plasma optical emission spectrum according to an embodiment of the disclosure.

FIG. 15 is a schematic view of a process flow of the method of testing a substance in a liquid under test through plasma optical emission spectrum according to an embodiment of the disclosure.

### DETAILED DESCRIPTION OF THE INVENTION

The technical features of the disclosure are illustrated by embodiments, depicted by drawings, and described below. Ordinal numbers, such as “first”, “second” and “third”, used herein are intended to distinguish components from each other rather than place limitations on the components themselves or indicate a specific sequence of the components. Unless a specific number is otherwise specified, the indefinite article “a/an” refers to one or more components.

To facilitate understanding of the object, characteristics and effects of this present disclosure, embodiments together with the attached drawings for the detailed description of the present disclosure are provided.

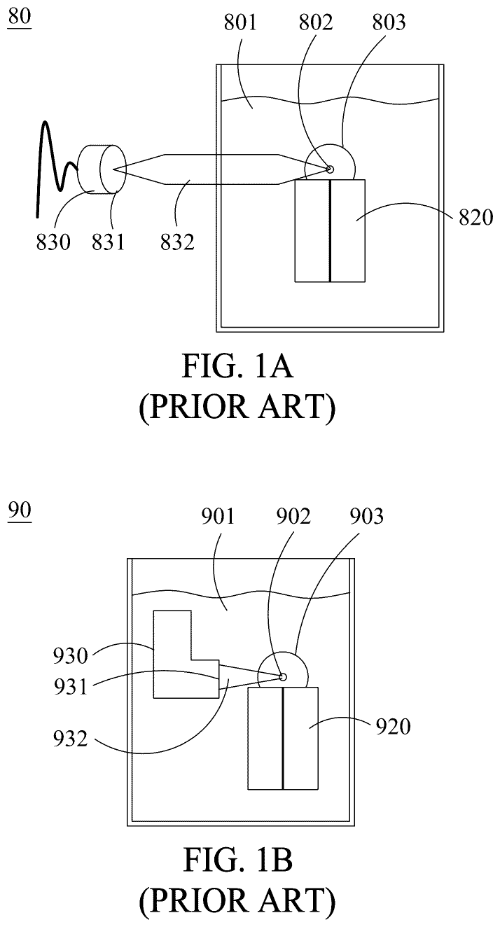

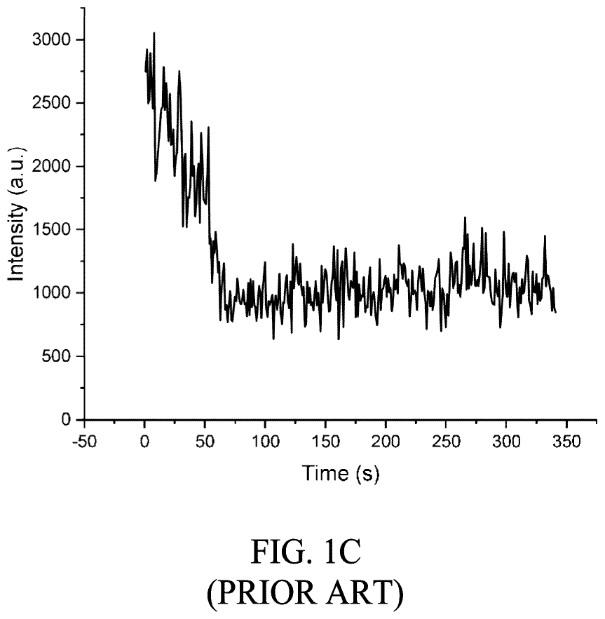

FIG. 1A is a schematic view of a conventional plasma testing device that receives light through a lens assembly outside a solution. FIG. 1B is a schematic view of a conventional plasma testing device that receives light through a lens assembly inside a solution. FIG. 1C is a schematic view of the trend of the intensity of signals obtained by a conventional plasma testing device that receives light through a lens assembly inside a solution.

Referring to FIG. 1A and FIG. 1B, according to prior art, a conventional testing device uses an aqueous solution plasma technique to analyze elements of a substance in an aqueous solution under test in two ways: with a light detection element disposed outside an aqueous solution to receive light through a lens assembly outside the solution (as shown in FIG. 1A); with a light detection element disposed in an aqueous solution to receive light through an underwater lens assembly (as shown in FIG. 1B).

Referring to FIG. 1A, a conventional plasma testing device 80 that receives light through a lens assembly disposed outside a solution has an electrode 820 immersed in a liquid under test 801 and has a light detection element 830 disposed outside the liquid under test 801 to prevent the liquid under test 801 from interfering with the light detection element 830 and causing loss thereto. To receive light precisely and effectively, a light-focusing component 831 is disposed at a light-receiving point of the light detection element 830 such that light signals of an optical emission spectrum of a plasma 802 produced by the electrode 820 can be effectively collected by the light detection element 830 to facilitate subsequent analysis of the optical emission spectrum of the plasma 802 through a spectrometer connected to the light detection element 830 so as to identify substances, for example, heavy metals, contained in the liquid under test 801.

However, before or during the process of producing the plasma 802, a lot of bubbles 803 are generated in the vicinity of the electrode 820 and the plasma 802 because of the applied energy. The appearance and disappearance of the bubbles 803 affects the optical path of a light-receiving region 832 determined by the light-focusing component 831. In addition to the appearance and disappearance of the bubbles 803, uncertainties, such as the variations in the size of the bubbles 803, the movement of the bubbles 803, and the optical characteristics of the reflection and refraction taking place at gas-liquid interfaces of the bubbles 803, lead to the reduction in the intensity and accuracy of signals associated with the optical emission spectrum of the plasma 802 and collected by the light detection element 830, causing interference with and difficulty in the subsequent analysis of substances. For the sake of clarity, FIG. 1A does not show a plurality of bubbles which differ in size and manifest behavioral uncertainty. In fact, bubbles which differ in size and manifest behavioral discrepancies are located in the light-receiving region 832.

Referring to FIG. 1B, a conventional plasma testing device 90 that receives light through a lens assembly inside a solution comprises a light detection element 930 and an electrode 920, both immersed in a liquid under test 901, in an attempt to mitigate the effect of a bubble 903 on a light-receiving region 932.

However, before or during the process of producing a plasma 902, a lot of bubbles 903 are generated in the vicinity of the electrode 920 and the plasma 902 and in the vicinity of the light detection element 930 and a light-focusing component 931 because of the applied energy. The appearance and disappearance of the bubbles 903 also affects the optical path of the light-receiving region 932 defined by the light-focusing component 931. In addition to the aforesaid issues, the bubbles 903 thus generated may attach to the surface of the light-focusing component 931 and thereby further affect light-receiving efficiency and accuracy.

Referring to FIG. 1C, there is show an intensity versus time graph about signals obtained by a light detection element of a conventional plasma testing device that receives light through a lens assembly inside a solution. Owing to the generation of a lot of bubbles and the resultant interference, the ongoing production of the plasma is not only accompanied by the generation of a lot of bubbles but also affects the light-receiving optical path; as a result, signal intensity decreases with time, markedly affecting the intensity and resolution of the collected signals of the optical emission spectrum in the plasma.

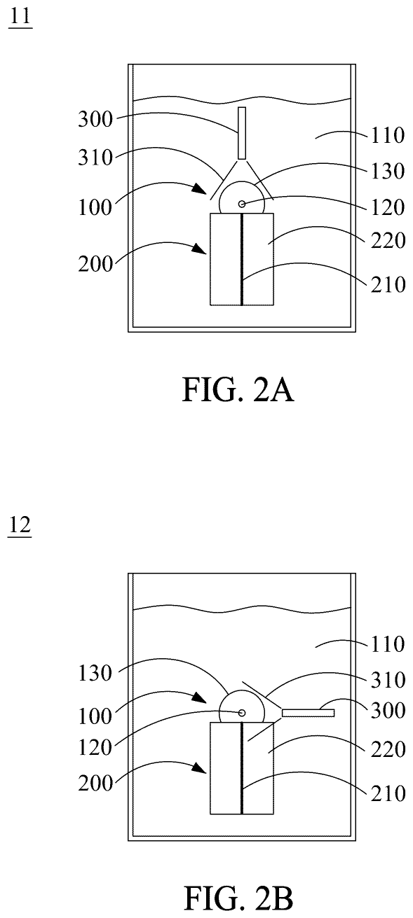

FIG. 2A is a schematic view of the device of testing a substance in a liquid under test through plasma optical emission spectrum according to an embodiment of the disclosure. FIG. 2B is a schematic view of the device of testing a substance in a liquid under test through plasma optical emission spectrum according to an embodiment of the disclosure.

Referring to FIG. 2A, to address the issue with the aforesaid bubble-induced signal intensity attenuation and resolution degradation, an aspect of the disclosure is a device 11 of testing a substance in a liquid under test through plasma optical emission spectrum. The device 11 comprises a sample region 100, an electrode 200 and a light detection element 300. The sample region 100 is adapted to contain a liquid under test 110.

At least a portion of the electrode 200 is located within the sample region 100 and adapted to be in contact with the liquid under test 110. The electrode 200 is adapted to produce a plasma 120 in the liquid under test 110 under an applied voltage, and the plasma 120 is located in a bubble 130 generated under the applied voltage. In an embodiment, the electrode 200 extends from outside and thus is partially immersed in the liquid under test 110 to produce the plasma 120 and the bubble 130 within the sample region 100 under the applied voltage. In an embodiment, the electrode 200 comprises a conductive portion 210 capable of conducting electricity and an insulating portion 220 capable of insulating and enclosing, whereas a free end (located within the sample region 100 and being in contact with the liquid under test 110) of the electrode 200 is, for example, a flat surface, a concave surface, and a convex surface, but the disclosure is not limited thereto. In a variant embodiment, a part of the conductive portion 210 is exposed to the liquid under test 110, and the other part of the conductive portion 210 is enclosed by the insulating portion 220. In an embodiment, the conductive portion 210 and the insulating portion 220 of the electrode 200 are, for example, coaxial cylinders. The conductive portion 210 is coaxially enclosed by the insulating portion 220. The conductive portion 210 within the sample region 100 is defined by a short radius, and the insulating portion 220 within the sample region 100 is defined by a long radius, defining an effective electrode region adapted to be in contact with the liquid under test 110 within the sample region 100 and produce the plasma 120 in the liquid under test 110; however, the abovementioned serves an illustrative purpose only. In fact, the conductive portion 210 and the insulating portion 220 of the electrode 200 can be of any shapes and are not necessarily coaxial as long as the insulating portion 220 partially encloses the conductive portion 210. With the electrode 200 being immersed in the liquid under test 110, the electrode 200 may have the conductive portion 210 but does not have the insulating portion 220. In an embodiment, the conductive portion 210 of the electrode 200 is made of platinum, and the insulating portion 220 of the electrode 200 is made of glass, allowing the electrode 200 to be made of glass platinum. The electrode 200 is a positive electrode, and its negative electrode is a silver wire in an embodiment, for example, silver wire (CAS: 7440-22-4) manufactured by Alfa Aesar, whereas the negative electrode is, for example, immersed in the liquid under test. In an embodiment, as opposed to its one end for being in contact with the liquid under test 110, the electrode 200 has the other end electrically connected to a power, for example, a power supply or a pulse generator, for supplying power of different voltages, intensities, periods, and pulse widths to enable the electrode 200 to produce the plasma 120.

The light detection element 300 is adapted to detect the optical emission spectrum generated from the plasma 120 in the bubble 130. The light detection element 300 is an optical fiber. No light-focusing component is present between the light detection element 300 and the plasma 120. No light-focusing component capable of focusing signals of the optical emission spectrum toward the light detection element 300 is present on the optical path from the optical emission spectrum generated from the plasma 120 to the light detection element 300.

Therefore, in an embodiment of the disclosure, the light detection element 300 is an optical fiber not having any light-focusing component, and its light-receiving end for receiving the optical emission spectrum generated from the plasma 120 does not have any light-focusing component, such as lens, microlens, and coupling connector, allowing the light detection element 300 to not only have a large light-receiving region 310 without being restricted to receiving light signals being focused toward a specific focus but also efficiently collect signals of the optical emission spectrum generated from the plasma 120 without being affected by the bubble 130. For the sake of explanation, the expression “an optical fiber not having any light-focusing component” is descriptive of “an optical fiber not having any light-focusing component but functioning as the light detection element 300 having a light-receiving end corresponding in position to the plasma 120.” In an embodiment, as opposed to its one end for detecting the plasma 120 in the liquid under test 110, the light detection element 300 has the other end electrically connected to a spectrometer to not only obtain information about the optical emission spectrum of the plasma 120, such as distribution of signal intensity and wavelength, but also further analyze and obtain information about the elements constituting the substances in the liquid under test 110. After the signals of the optical emission spectrum generated from the plasma 120 have entered the light detection element 300 (i.e., spectrum signals have been collected by the light detection element 300,) the spectrum signals are transmitted by an appropriate optical component (not shown). For example, after the signals of the optical emission spectrum generated from the plasma 120 have entered the light detection element 300, the light signals which have already been collected and received by the light detection element 300 are effectively transmitted to the spectrometer by a light-focusing component. The optical fiber is provided in the form of a bare optical fiber or an optical fiber with a protective enclosing layer in order to operate in different usage environments, meet user needs, satisfy size requirements.

All components shown in the accompanying drawings and the liquid under test 110 are disposed in a processing chamber, any container, or any device or are directly located in natural waterbodies. For the sake of the clarity of the accompanying drawings, the accompanying drawings omit a means of connecting or fixing the electrode 200 and the light detection element 300 to external signals, which may be replaced by any well-known means of fixation or means of signal delivery, for example, transmitting signals by a fixation tool manufactured by CNC processing or through wired connection or wireless communication.

Referring to FIG. 2A, the device 11 of testing a substance in a liquid under test through plasma optical emission spectrum directly uses an optical fiber not having any light-focusing component to function as the light detection element 300 for detecting the plasma 120 produced by the electrode 200. Since no light-focusing component, such as a lens, is present, the light-receiving region 310 of the light detection element 300 and its scope are effectively enlarged. Since no light-focusing component is present, the light-receiving scope of the light detection element 300 is not excessively focused toward an optical focus. Therefore, despite the random generation, movement and alteration of a lot of bubbles 130, none of the bubbles 130 greatly affects the position of the focus to otherwise cause the deviation of the light-receiving scope from the actual position of the plasma 120, allowing the light signals generated from the plasma 120 to be effectively collected to greatly enhance the signal intensity, signal resolution and accuracy of the detected optical emission spectrum of the plasma 120.

The device 12 of testing a substance in a liquid under test through plasma optical emission spectrum as shown in FIG. 2B differs from the device 11 of testing a substance in a liquid under test through plasma optical emission spectrum as shown in FIG. 1A in terms of the position of the light detection element 300. As shown in FIG. 1A, the light detection element 300 is disposed above the electrode 200 squarely, positioned in the direction of the normal of the contact surface between the electrode 200 and the liquid under test 110, and disposed in the axial direction (because it is disposed in the axial direction of the line connecting the electrode 200 and the plasma 120 produced by the electrode 200.) As shown in FIG. 2B, the light detection element 300 is disposed in the horizontal direction at the point where the plasma 102 is produced by the electrode 200, positioned in a direction perpendicular to the normal of the contact surface between the electrode 200 and the liquid under test 110, and disposed in the radial direction (because it is disposed in the radial direction of the line connecting the electrode 200 and the plasma 120 produced by the electrode 200.) The difference between positioning the light detection element 300 in the radial direction and positioning the light detection element 300 in the axial direction is as follows: in general, the bubbles 130 move upward, and the light detection element 300 disposed in the axial direction are more likely to be affected by the floating, approaching bubbles 130; thus, when both the light detection element 300 and the electrode 200 are disposed inside the liquid under test 110 but separated by a long distance, the light detection element 300 disposed in the radial direction are less likely to be affected by the floating bubbles 130. However, given a short distance between the light detection element 300 and the electrode 200, the light detection element 300 disposed in the axial direction is less likely to be affected by the background and thus manifests satisfactory signal intensity and signal stability (to be described later).

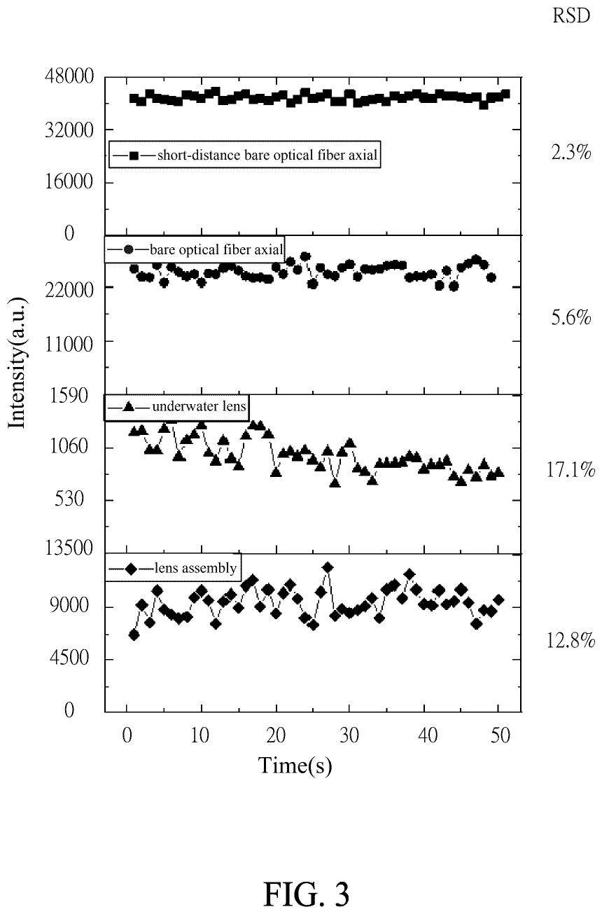

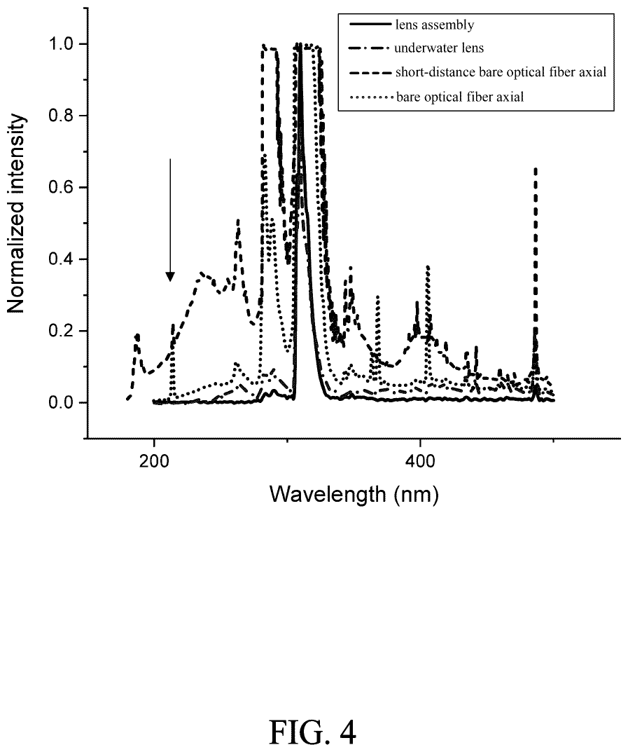

FIG. 3 is a schematic view of comparing the intensity of signals obtained (by the conventional plasma testing device 80 that receives light through a lens assembly outside a solution and the conventional plasma testing device 90 that receives light through a lens assembly inside a solution as shown in FIG. 1A and FIG. 1B respectively) according to the prior art with the intensity of signals obtained by the device (denoted by 11 in FIG. 2A and disposed axially, and denoted by 12 in FIG. 2B and disposed radially) of testing a substance in a liquid under test through plasma optical emission spectrum according to an embodiment of the disclosure. FIG. 4 is a schematic view of comparing the spectrum distribution obtained (by the conventional plasma testing device 80 that receives light through a lens assembly outside a solution and the conventional plasma testing device 90 that receives light through a lens assembly inside a solution as shown in FIG. 1A and FIG. 1B respectively) according to the prior art with the spectrum distribution obtained by the device (denoted by 11 in FIG. 2A and disposed axially, and denoted by 12 in FIG. 2B and disposed radially) of testing a substance in a liquid under test through plasma optical emission spectrum according to an embodiment of the disclosure.

Referring to FIG. 3, there is shown a schematic view of comparing the signal intensity and its relative standard deviation (RSD) of the lens assembly and the underwater lens provided according to the prior art with the signal intensity and its relative standard deviation (RSD) of the optical fiber radial and the short-distance optical fiber axial provided according to an embodiment of the disclosure.

As shown in FIG. 3, when the conventional lens assembly disposed outside a liquid under test functions as a light detection element, its signal intensity ranges from 6000 a.u. to 13000 a.u., with an RSD of 12.8%; although the optical component itself is not directly affected by the liquid under test, signals received by the lens assembly manifest intense changes and poor stability because of the generation and alteration of the bubbles, unequal sizes of bubbles, movements of bubbles, and optical characteristics of gas-liquid interfaces.

As shown in FIG. 3, when the conventional underwater lens assembly disposed inside the liquid under test functions as a light detection element, its signal intensity ranges from 550 a.u. to 1500 a.u., with an RSD of 17.1%; although the optical component is directly disposed underwater in an attempt to mitigate the effect of the bubbles on the optical path, not only is the vicinity of the underwater lens assembly directly confronted with the interference from a lot of bubbles, but the optical path for collecting plasma luminescence is also affected. Since the underwater lens assembly is positioned underwater, the bubbles increasingly attach to the surface of the underwater lens assembly as time passes, further affecting the light-receiving efficiency and accuracy. As shown in the diagram, the signal intensity of the underwater lens assembly attenuates as plasma generation time passes, markedly affecting the intensity and resolution of the collected signals of the optical emission spectrum in the plasma.

Referring to FIG. 3, when an optical fiber not having any light-focusing component functions as a light detection element and receives light in the radial direction in an embodiment of the disclosure, its signal intensity ranges from 21000 a.u. to 27000 a.u., with an RSD of 5.6%, indicating that receiving light directly with an optical fiber radial instead of a light-focusing component is conducive to effective collection of light emitted from the plasma. Although the conventional lens assembly and underwater lens also receive light radially, their performance is unsatisfactory. By contrast, unlike lens focusing, receiving light with an optical fiber radial (denoted by 12 in FIG. 2B) according to the disclosure does not markedly alter the light-receiving scope because of the bubbles but markedly reduces the interference from or effect of the bubbles to obtain high-intensity and high-stability light signals of plasma optical emission spectrum.

Referring to FIG. 3, when an optical fiber not having any light-focusing component functions as a light detection element and receives light in the short-distance axial direction in an embodiment of the disclosure, its signal intensity ranges from 38000 a.u. to 46000 a.u., with an RSD of 2.3%, indicating that receiving light with a short-distance optical fiber axial is conducive to optimization of collection of light emitted from the plasma. It is because an optical fiber approaching the electrode not only has the advantage of an optical fiber not having any light-focusing component, i.e., not being subjected to the effect of altering a focus through the bubbles, but also shortens the distance between the light-receiving end and the plasma to reduce interference between the optical paths. Furthermore, when the light-receiving end of the optical fiber not having any light-focusing component but functioning as a light detection element is sufficiently close to the electrode and the plasma produced by the electrode, the light-receiving end of the optical fiber is directly positioned in a bubble containing the plasma to minimize the effect of the bubble on light receiving, greatly enhancing the intensity and stability of light signals of plasma optical emission spectrum.

FIG. 4 is a schematic view of comparing the spectrum distribution obtained from comparison results shown in FIG. 3 respectively. As shown in FIG. 4, when the spectrum distributions of the conventional lens assembly, the conventional underwater lens as well as the short-distance optical fiber axial and the optical fiber radial not having any light-focusing component according to the disclosure are examined against identical criteria, regardless of whether the wavelength is less than 300 nm approximately or greater than 330 nm approximately. Except for one single wave peak, the equalized signal intensity of the short-distance optical fiber axial and the optical fiber radial not having any light-focusing component according to the disclosure is obviously higher than the signal intensity of the conventional lens assembly and underwater lens at each wavelength, indicating the obvious advantages of the device of testing a substance in a liquid under test through plasma optical emission spectrum and using an optical fiber not having any light-focusing component to function as a light detection element according to the disclosure. In an embodiment, compared with the light receiving technique applied to the conventional lens assembly and underwater lens, using a short-distance axial optical fiber and a radial optical fiber not having any light-focusing component to function as a light detection element for receiving light according to an embodiment of the disclosure can test signals emitted from the liquid under test containing zinc (Zn) (indicated by arrows) on the spectrum better.

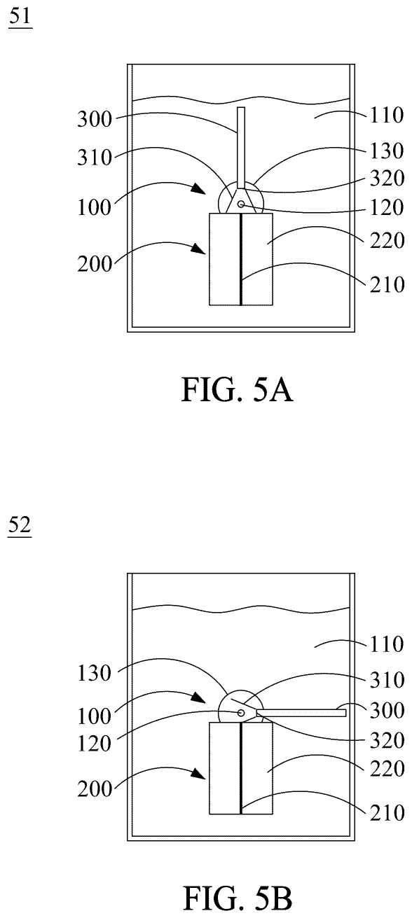

FIG. 5A depicts the device of testing a substance in a liquid under test through plasma optical emission spectrum according to an embodiment of the disclosure. FIG. 5B depicts the device of testing a substance in a liquid under test through plasma optical emission spectrum according to an embodiment of the disclosure.

The device 51 of testing a substance in a liquid under test through plasma optical emission spectrum as shown in FIG. 5A is distinguished from the device 11 of testing a substance in a liquid under test through plasma optical emission spectrum as shown in FIG. 2A by technical features as follows: at least a portion of the light detection element 300 is located within the sample region 100; and at least a portion of the light detection element 300 comprises a light-receiving end 320 adapted to be located in the bubble 130 to detect the optical emission spectrum generated from the plasma 120 in the bubble 130.

One end of the light detection element 300 is directly located within the sample region 100, and the light-receiving end 320 of the light detection element 300 is directly positioned in the vicinity of the electrode 200. The light-receiving end 320 of the light detection element 300 is close to the position of the plasma 120 produced by the electrode 200 and thus is positioned within the scope of the bubble 130 having therein the plasma 120 to not only minimize the effect of the bubble 130 and the interface thereof on light receiving but also minimize the effect of the liquid phase (for example, water), which a portion of the optical path passes through, on partial spectrum absorption in light signals, allowing the light detection element 300 to collect the light signals of the plasma 120 through the light-receiving end 320 in the bubble 130. With the light-receiving end 320 of the light detection element 300 being sufficiently close to the electrode 200, advantages thus achieved are as follows: sparing the light detection element 300 any interference from or effect of the other bubbles; minimizing the noise-related impact or effect of the other gas-liquid interfaces on the background; preventing part of the wave band of the light signals from the plasma 120 from being absorbed by passing water; maximizing the complete collection of the light signals of the optical emission spectrum generated from the plasma 120; and maximizing the optical path and light-receiving stability. When the light-receiving end 320 of the light detection element 300 is sufficiently close to the electrode 200, the bubble 130 exists steadily throughout the course of plasma luminescence of the plasma 120, precluding the creation of new bubbles or the interference between the light-receiving end 320 and the plasma 120, regardless of whether the bubble 130 forms a film layer on the surface of the light-receiving end 320. Since the bubble 130 is created and vanishes according to the voltage applied by the electrode 200 and its duration or any other disturbance, the bubble 130 is created to grow and vanish outside the time period in which the light detection element 300 detects the optical emission spectrum generated from the plasma 120, for example, before and after the production of the plasma 120, to alter its interface, cleaning the surface of the nearby light-receiving end 320 of the light detection element 300.

The device 52 of testing a substance in a liquid under test through plasma optical emission spectrum as shown in FIG. 5B is distinguished from the device 12 of testing a substance in a liquid under test through plasma optical emission spectrum as shown in FIG. 2B by technical features as follows: at least a portion of the light detection element 300 is located within the sample region 100; and at least a portion of the light detection element 300 comprises a light-receiving end 320 adapted to be located in the bubble 130 to detect the optical emission spectrum generated from the plasma 120 in the bubble 130.

The light detection element 300 of the device 51 of testing a substance in a liquid under test through plasma optical emission spectrum as shown in FIG. 5A is axially positioned and thus positioned in front of the electrode 200. By contrast, the light detection element 300 of the device 52 of testing a substance in a liquid under test through plasma optical emission spectrum as shown in FIG. 5B is radially positioned on the lateral side of the electrode 200. Since the light-receiving end 320 of the light detection element 300 of each of the device 51 and the device 52 is sufficiently close to the position of the plasma 120 produced by the electrode 200 and thereby is positioned in the bubble 130 having therein the plasma 120 produced to effectively and optimally collect the light signals of the optical emission spectrum of the plasma 120 without being subjected to the interference from the other bubbles or the optical path. When the light-receiving end 320 of the light detection element 300 is sufficiently close to the electrode 200 and thereby is positioned in the bubble 130, the light detection element 300 (denoted by 51 in FIG. 5A) axially positioned is not affected by the ascent of the bubble 130 and the resultant signal interference for two reasons as follows: when the light detection element 300 is sufficiently close to the electrode 200, its spatial limitation reduces the space and probability of the ascent and escape of the bubble 130; even if the bubble ascends, the ascent will not affect the light-receiving region 310. A comparison of the embodiment of the light detection element 300 (denoted by 51 in FIG. 5A) axially positioned and the embodiment of the light detection element 300 (denoted by 52 in FIG. 5B) radially positioned shows that the light detection element 300 axially positioned not only surpasses the light detection element 300 radially positioned in signal intensity and stability but also holds the bubble 130 to render the bubble 130 less likely to escape or move than the light detection element 300 radially positioned and thereby achieves higher signal intensity and stability, because the light detection element 300 axially positioned is unlikely to be affected by the other factors of the background.

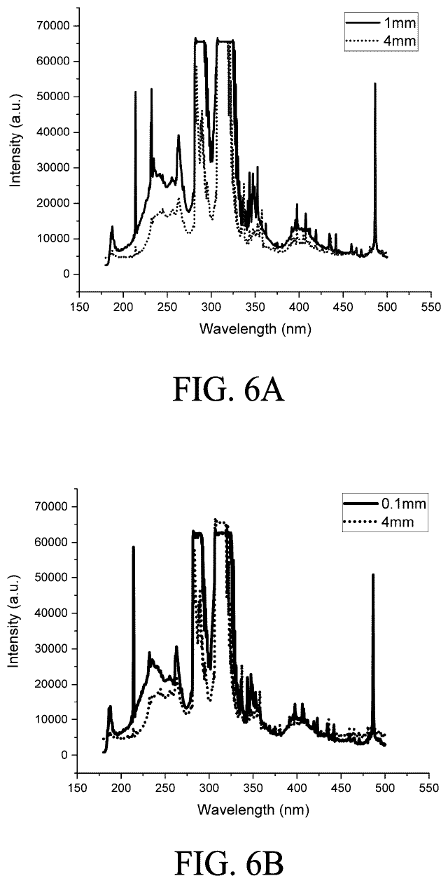

FIG. 6A and FIG. 6B are schematic views of comparing the spectrum intensity obtained as a result of different distances between the light detection element and the electrode according to an embodiment of the disclosure respectively. Referring to FIG. 6A, there is shown a schematic view of comparing the spectrum intensity obtained as a result of a distance of 1 mm (solid line) and a distance of 4 mm (dotted line) between the light-receiving end 320 of the light detection element 300 and the electrode 200 in an embodiment when the light detection element 300 is disposed in the axial direction (denoted by 51 in FIG. 5A). Referring to FIG. 6B, there is shown a schematic view of comparing the spectrum intensity obtained as a result of a distance of 0.1 mm (solid line) and a distance of 4 mm (dotted line) between the light-receiving end 320 of the light detection element 300 and the electrode 200 in an embodiment when the light detection element 300 is disposed in the axial direction (denoted by 51 in FIG. 5A). In the embodiments, the plasma 120 is produced under a voltage of 600 V, and the electrode 200 has an effective size (hereinafter the conductive portion 210 in FIG. 5A) of 0.3 mm, pulse on-time of 10 ms, and pulse off-time of 10 ms. As shown in FIG. 6A and FIG. 6B, given a short distance between the light detection element 300 and the electrode 200, the light detection element 300 axially positioned is very close to the plasma 120 to not only spare the light detection element 300 any interference from the bubble 130 but also prevent the light signals from being absorbed by passing water (or the liquid under test 110), demonstrating the obvious advantages of allowing the light detection element 300 to be close to the electrode 200.

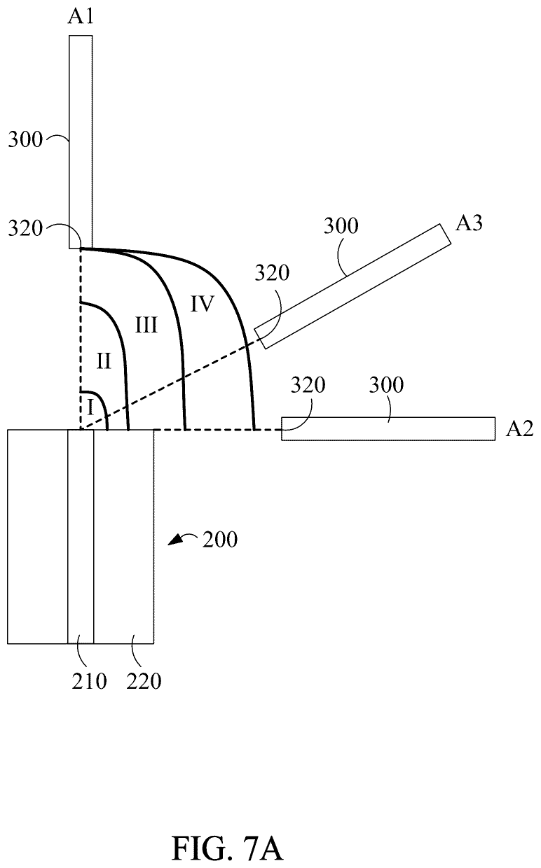

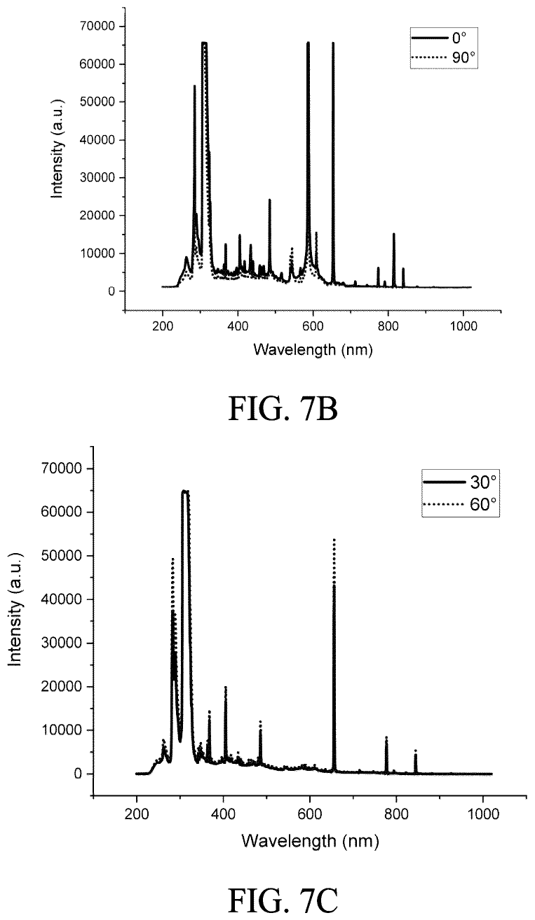

FIG. 7A is a schematic view of different angles between the light detection element and the electrode according to an embodiment of the disclosure. FIG. 7B is a schematic view of comparing the spectrum intensity obtained as a result of different angles between the light detection element and the electrode according to an embodiment of the disclosure. FIG. 7C is a schematic view of comparing the spectrum intensity obtained as a result of different angles between the light detection element and the electrode according to an embodiment of the disclosure.

FIG. 7A depicts, on an exemplary basis, the light detection element 300 positioned at a first angle A1, second angle A2, and third angle A3 relative to the electrode 200. The light detection element 300 is oriented at the first angle A1 to the normal of the contact surface between the electrode 200 and the liquid under test 110, with an included angle of 0° defined between the first angle A1 and the normal of the contact surface; thus, when the light detection element 300 is oriented at the first angle A1, the light detection element 300 lies in the axial direction (i.e., the aforesaid axial direction) of the electrode 200 relative to the plasma 120. The light detection element 300 is oriented at the second angle A2 to the normal of the contact surface between the electrode 200 and the liquid under test 110, with an included angle of 90° defined between the second angle A2 and the normal of the contact surface; thus, when the light detection element 300 is oriented at the first angle A2, the light detection element 300 lies in the radial direction (i.e., the aforesaid radial direction) of the electrode 200 relative to the plasma 120. The light detection element 300 is oriented at the third angle A3 to the normal of the contact surface between the electrode 200 and the liquid under test 110, with an included angle ranging between the first angle A1 and the second angle A2 and defined between the third angle A3 and the normal of the contact surface.

Referring to FIG. 7A, when the light detection element 300 is oriented at different angles, four different ranges, namely range I, range II, range III and range IV, of a light-receiving distance of the light detection element 300 are defined. Range I is defined as a distance nearest to the electrode 200 and nearest to the plasma 120; however, when the light detection element 300 is overly close to the electrode 200, the overly short distance is likely to interfere with the generation of the bubble 130 and the plasma 120, impeding the collection of the optical emission spectrum of the plasma 120. Range II is defined as the best light-receiving distance of the light detection element 300 to achieve an advantage as follows: when the light-receiving end 320 of the light detection element 300 is sufficiently close to the electrode 200 but not sufficient to affect the generation of the bubble 130 or the plasma 120, the light signals of the optical emission spectrum generated from the plasma 120 can be effectively, directly collected within the scope of the bubble 130 but are neither subjected to the interference from the generation or alteration of the other bubbles nor subjected to the absorption-related interference from water or the liquid under test, greatly enhancing light-receiving intensity and accuracy. Range III is defined as the range during which the light-receiving end 320 is increasingly farther from the electrode 200 and the plasma 120 generated by the electrode 200 to achieve an advantage as follows: the light-receiving end 320 of the light detection element 300 has already left the bubble 130 which contains the plasma 120 and thus is being affected by the other bubbles or liquid; however, with the optical fiber functioning as the light detection element 300, the light signals of the optical emission spectrum generated from the plasma 120 remain unaffected by a focus change and the otherwise resultant interference with light receiving, allowing the light signals to be effectively collected and received by the optical fiber. Range IV has an advantage as follows: the light-receiving end 320 moving further away increases the effect of the absorption of the light signals of the optical emission spectrum generated from the plasma 120 by water or the liquid under test even though the other bubbles are unlikely to attach to the light detection element 300; however, unlike its conventional counterparts, the optical fiber is effective in maintaining a certain degree of effective light receiving and thus remains unaffected by a conventional focus change and the otherwise resultant intense interference.

In an embodiment, when the light detection element 300 is oriented at the first angle A1 and disposed in the axial direction relative to the electrode 200, preferably, at least a portion of the light detection element 300 is located within the sample region 100 and comprises a light-receiving end 320, with the light-receiving end 320 adapted to be located within the scope of the bubble 130, i.e., range II, because of the distance between the light-receiving end 320 and the electrode 200. Therefore, the optimal light-receiving distance of the light detection element 300 oriented at the first angle A1 falls within the range II of 0.1 mm to 4 mm, the range I less than 0.1 mm, and the range III greater than 4 mm.

In an embodiment, when the light detection element 300 is oriented at the second angle A2 and disposed in the radial direction relative to the electrode 200, preferably, at least a portion of the light detection element 300 is located within the sample region 100 and comprises a light-receiving end 320, with the light-receiving end 320 adapted to be located within the scope of the bubble 130, i.e., the range II, because of the distance between the light-receiving end 320 and the electrode 200. Therefore, the light detection element 300 oriented at the second angle A2 and positioned on the lateral side of the electrode 200 rather than above the electrode 200 shortens the distance required for maintaining the stability of the bubble 130 and thus has an optimal light-receiving distance that falls within the range II of 0.05 mm to 3.5 mm, the range I less than 0.05 mm, the range III of 3.5 mm to 4.5 mm, and the range IV greater than 4.5 mm.

In an embodiment, when the light detection element 300 is oriented at the third angle A3 and disposed in between the axial direction and the radial direction relative to the electrode 200, preferably, at least a portion of the light detection element 300 is located within the sample region 100 and comprises a light-receiving end 320, with the light-receiving end 320 adapted to be located within the scope of the bubble 130, i.e., the range II, because of the distance between the light-receiving end 320 and the electrode 200. Therefore, the optimal light-receiving distance of the light detection element 300 oriented at the third angle A3 and positioned between the top side and the lateral side of the electrode 200 to allow the distance required for maintaining the stability of the bubble 130 to be close to the radial direction falls within the range II of 0.05 mm to 3.5 mm, the range I less than 0.05 mm, the range III of 3.5 mm to 4.5 mm, and the range IV greater than 4.5 mm.

Referring to FIG. 7B and FIG. 7C, there are shown schematic views of comparing the spectrum intensity obtained as a result of different angles between the light detection element and the electrode according to an embodiment of the disclosure, indicating no marked differences in signal features at different wavelengths and intensity of spectrum distribution and manifesting stable, clear resolution and signal intensity of feature wave peaks at different wavelengths when light receiving is carried out with the light detection element 300 at different angles under the same condition, with the distances between the light detection element 300 and the electrode 200 falling within range II.

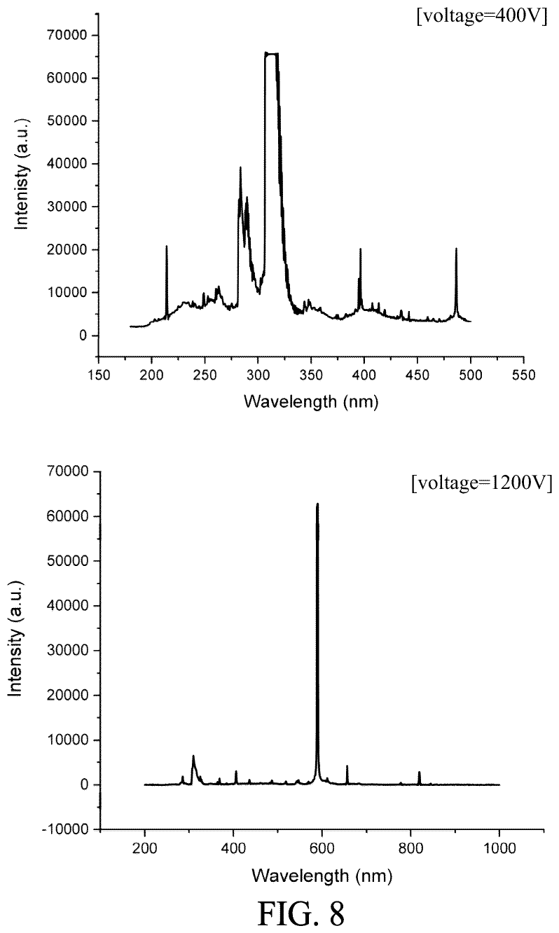

FIG. 8 are schematic views of the spectrum intensity obtained by applying different applied voltages to the electrode according to an embodiment of the disclosure.

Referring to FIG. 8, there are shown the distributions of the spectrum intensity obtained by applying different applied voltages to the electrode according to an embodiment of the disclosure. The upper diagram in FIG. 8 depicts the distribution of the spectrum intensity obtained by applying an applied voltage of 400 V. The lower diagram in FIG. 8 depicts the distribution of the spectrum intensity obtained by applying an applied voltage of 1200 V. In addition to the aforesaid two criteria applicable to the upper and lower diagrams, the distribution of the spectrum intensity obtained and depicted by the upper and lower diagrams is achieved with an electrode size of 0.3 mm and 0.8 mm respectively, pulse application on-time of 10 ms and 0.2 ms respectively, and pulse application off-time of 1 ms and 50 ms respectively. As shown in FIG. 8, when voltages of 400 V and 1200 V are applied respectively, essential features of different wavelengths of the plasma optical emission spectrum thus generated and received can still be discerned.

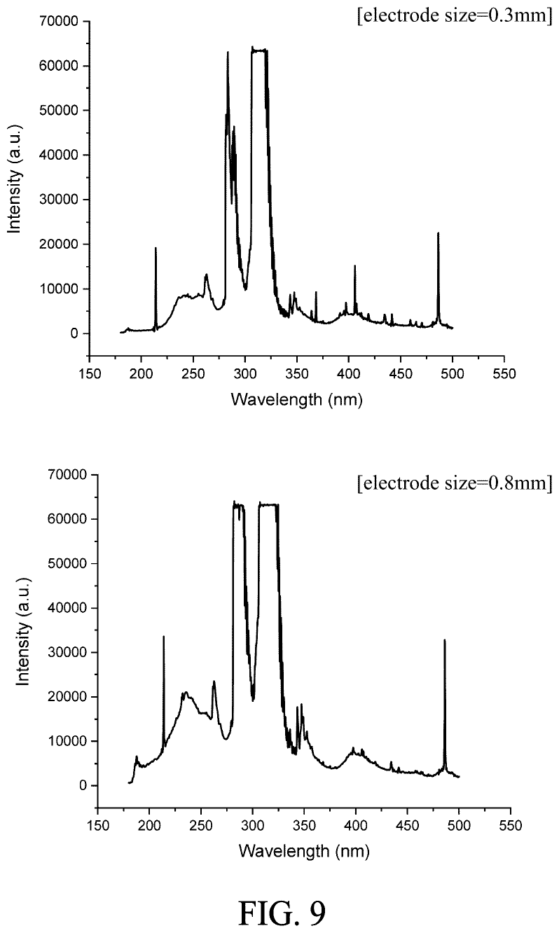

FIG. 9 are schematic views of the spectrum intensity obtained by using different electrode sizes according to an embodiment of the disclosure.

Referring to FIG. 9, there are shown the distributions of the spectrum intensity obtained by using different electrode sizes according to an embodiment of the disclosure. The upper diagram in FIG. 9 depicts the distribution of the spectrum intensity obtained by using an electrode size of 0.3 mm. The lower diagram in FIG. 9 depicts the distribution of the spectrum intensity obtained by using an electrode size of 0.8 mm. In addition to the aforesaid two criteria applicable to the upper and lower diagrams, the distribution of the spectrum intensity obtained and depicted by the upper and lower diagrams is achieved by applying an applied voltage of 650 V, pulse application on-time of 10 ms, and pulse application off-time of 10 ms. As shown in FIG. 9, when electrode sizes of 0.3 mm and 0.8 mm are used respectively, essential features of different wavelengths of the plasma optical emission spectrum thus generated and received can still be discerned.

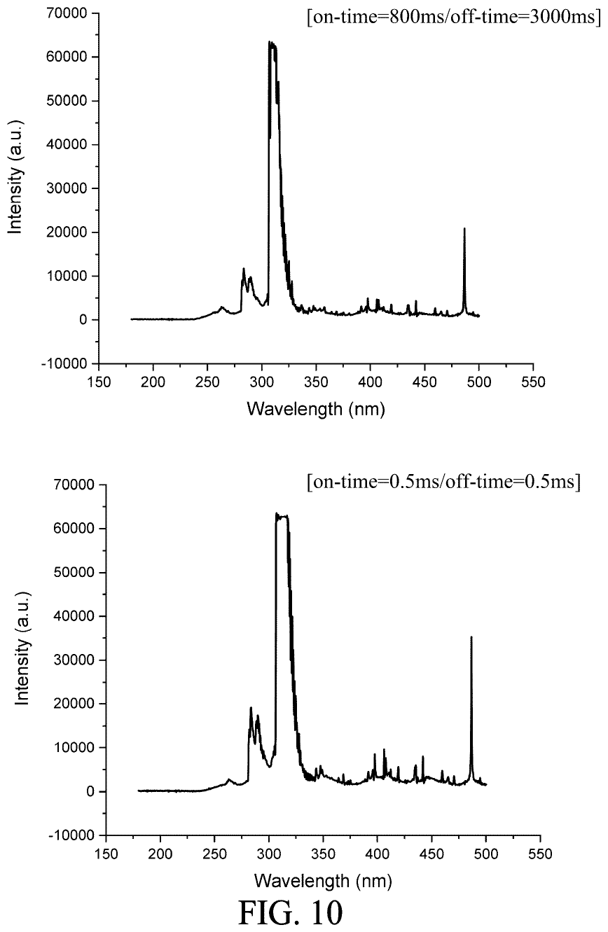

FIG. 10 are schematic views of the spectrum intensity obtained by applying different pulse times to the electrode according to an embodiment of the disclosure.

Referring to FIG. 10, there are shown the distributions of the spectrum intensity obtained by applying different voltages of different pulse times to the electrode according to an embodiment of the disclosure. The upper diagram in FIG. 10 depicts the distribution of the spectrum intensity obtained by applying a voltage of pulse on-time of 800 ms and pulse off-time of 3000 ms. The lower diagram in FIG. 10 depicts the distribution of the spectrum intensity obtained by applying a voltage of pulse on-time of 0.5 ms and pulse off-time of 0.5 ms. In addition to the aforesaid two criteria applicable to the upper and lower diagrams, the distribution of the spectrum intensity obtained and depicted by the upper and lower diagrams is achieved with a voltage of 540 V and an electrode size of 0.3 mm. As shown in FIG. 10, when voltages of pulse on-time of 800 ms, pulse off-time of 3000 ms, pulse on-time of 0.5 ms, and pulse off-time of 0.5 ms are applied respectively, essential features of different wavelengths of the plasma optical emission spectrum thus generated and received can still be discerned.

Therefore, the device of testing a substance in a liquid under test through plasma optical emission spectrum, as provided in an embodiment of the disclosure, is operated under criteria as follows: a voltage of 300 V to 1200 V, an electrode size of 0.1 mm to 1 mm, pulse on-time of 0.01 ms to 800 ms, a distance of 0.05 mm to 10 mm between the light detection element and the electrode, and a light receiving angle of 0° to 90° of the light detection element relative to the electrode. Not only can essential features of different wavelengths of the plasma optical emission spectrum generated under the aforesaid criteria and received still be discerned, but the objectives and advantages of testing a substance in a liquid under test through plasma optical emission spectrum are also achieved.

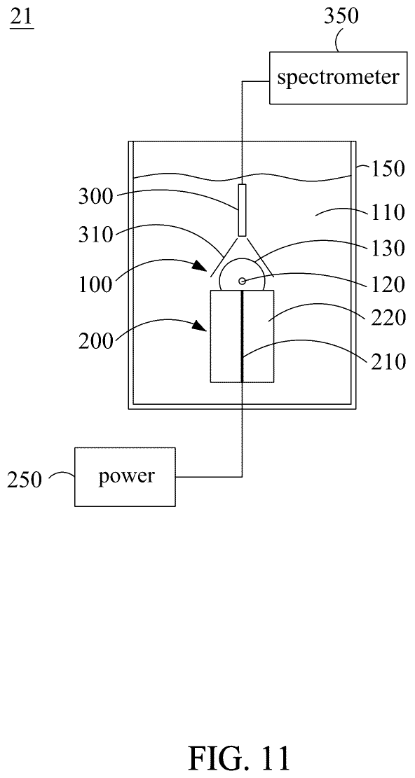

FIG. 11 is a schematic view of the system of testing a substance in a liquid under test through plasma optical emission spectrum according to an embodiment of the disclosure.

The system of testing a substance in a liquid under test through plasma optical emission spectrum according to an embodiment of the disclosure comprises the device 11, 12, 51, 52 of testing a substance in a liquid under test through plasma optical emission spectrum in the aforesaid embodiments, a sample chamber 150, a spectrometer 350 and a power 250.

The sample chamber 150 is configured to retain the electrode 200 and the light detection element 300 and thus form a sample region 100 between the electrode 200 and the light detection element 300 and adapted to receive the liquid under test 110. In an embodiment, the sample chamber 150 comprises a waterproof tank for receiving the liquid under test 110 and has an opening, pipeline, and valve through which the liquid under test 110 is admitted. In another embodiment, the sample chamber 150 is a simple rigid support which the electrode 200 and the light detection element 300 are mounted on, for example, a framework integrally formed by CNC processing and adapted to retain and fix the electrode 200 and the light detection element 300 in place. In another embodiment, the sample chamber 150 further has an adjustable supporting structure, a linear or annular rail, for example, for adjusting the angle and distance of the light detection element 300 relative to the electrode 200, a snap-engaging structure, and an engaging slot with predetermined multiple angles and multiple distances. In another embodiment, the sample chamber 150 is not a container for containing a liquid but is a structure for fixing the electrode 200 and the light detection element 300 in place and enabling the electrode 200 and the light detection element 300 to be directly delivered to and immersed in any waterbodies to test a substance in a liquid, dispensing with the need to sample, transport and input a liquid to a device or system.

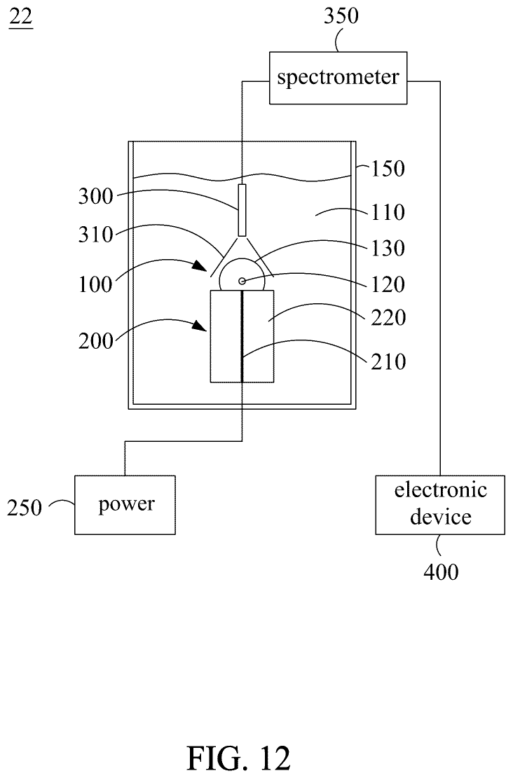

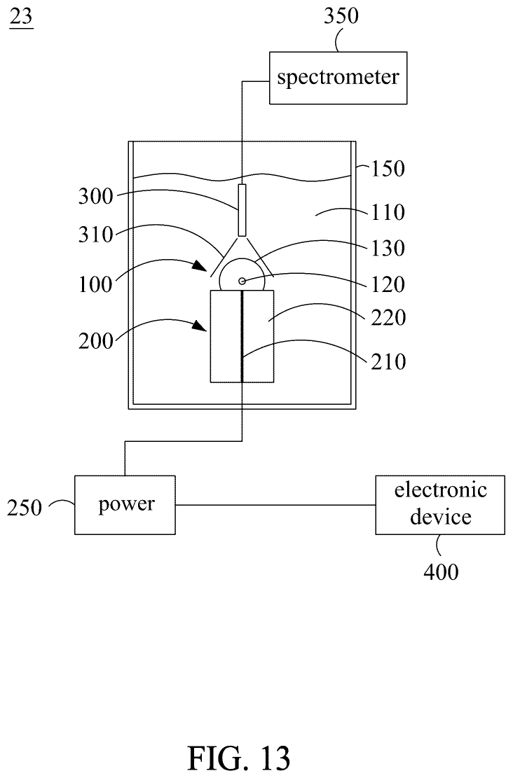

The spectrometer 350 is configured to be coupled to the light detection element 300 and configured to analyze the optical emission spectrum generated from the plasma 120, disposed in the bubble 130, and detected by the light detection element 300 so as to not only obtain information, such as wave peak signals and feature distribution of signal intensity and wavelength, pertaining to the optical emission spectrum of the plasma 120 but also obtain information about the elements of the substance in the liquid under test 110. In an embodiment, the light detection element 300 undergoes optical communication through any optical method and the spectrometer 350, for example, directly connects to the spectrometer 350 not via any other optical components, and continues to transmit light signals to the spectrometer 350 through the original optical fiber or transmit light signals to the spectrometer 350 through any other required optical components, for example, a lens and a compressing-expanding unit, allowing the spectrometer 350 to receive complete signals from the light detection element 300. The connection lines of the light detection element 300 and the spectrometer 350 and therebetween in FIG. 11, FIG. 12, and FIG. 13 serve a schematic purpose but do not denote actual physical and spatial dimensions differences and the dimensions themselves. The dimensions and ratios of the bubble 130, the plasma 120, the electrode 200 and the light detection element 300 shown in the accompanying drawings serve an illustrative purpose, and their dimensions, distances, ratios and angles as shown in the accompanying drawings are illustrative rather than restrictive of the disclosure.

The power 250 is configured to be coupled to the electrode 200 and more particularly coupled to the conductive portion 210 of the electrode 200. The power 250 is configured to provide an applied voltage to the electrode 200 to enable the electrode 200 to produce the plasma 120 and the bubble 130 in the liquid under test 110. In an embodiment, the power 250 is a power supply, function generator, pulse generator, or high voltage function generator for providing electrical energy in terms of different voltages, intensities, periods, and pulse widths to enable the electrode 200 to produce the plasma 120. The connection lines of the electrode 200 and the power 250 and therebetween in FIG. 11, FIG. 12, and FIG. 13 serve a schematic purpose but do not denote actual physical and spatial dimensions differences and distances, nor do they denote actual size ratios.

FIG. 12 depicts a system 22 of testing a substance in a liquid under test through plasma optical emission spectrum according to an embodiment of the disclosure. FIG. 13 depicts a system 23 of testing a substance in a liquid under test through plasma optical emission spectrum according to an embodiment of the disclosure.

Referring to FIG. 12 and FIG. 13, the system 22 of testing a substance in a liquid under test through plasma optical emission spectrum according to an embodiment of the disclosure further comprises an electronic device 400. The electronic device 400 is configured to electrically connect to the spectrometer 350 and is configured to further analyze, through the spectrometer 350, the information about the optical emission spectrum generated from the plasma 120.

In an embodiment, the electronic device 400 is further configured to be signal-connected to an external device (not shown) to provide an analysis result about an optical emission spectrum of the liquid under test 110 to the external device in real time. In an embodiment, the external device further connects to any other external electronic devices, such as a user's computer or cellphone, or a monitoring system dedicated to a family, company, plant or government department, to provide information about a test result of the liquid under test 110 to the monitoring system in real time. If an abnormal substance, such as a heavy metal or any other element regarded as harmful under an environmental protection law, in the liquid under test 110 is detected, all users and all monitoring systems will be informed of the detection result in real time to address the issue with the contamination of related waterbodies timely. The device and system of testing a substance in a liquid under test through plasma optical emission spectrum according to the disclosure not only perform a liquid test or water quality test on a case-by-case basis, intermittently and irregularly but also perform a liquid test or water quality test on a fixed-address, long-term basis in real time or not in real time, being applicable to a wide variety of scenes and sites.

In an embodiment, the system 23 of testing a substance in a liquid under test through plasma optical emission spectrum also comprises the electronic device 400 configured to electrically connect to the power 250 and configured to set a parameter of an applied voltage through the power 250 to adjust the characteristics of the plasma 120 in the liquid under test 110, for example, the pulse width, period, intensity and voltage applied to the electrode 200. In an embodiment, the electronic device 400 is not necessarily an additional apparatus but a simple circuit module, microprocessor or IC circuit additionally disposed at the spectrometer 350 or the power 250. By being connected to the electronic device 400, it is feasible for the spectrometer 350 and the power 250 to undergo programmable control each, undergo integrated programmable control together, and undergo remote programmable control, performing system information integration, automated controls, smart monitoring, and big data statistics. In an embodiment, the electronic device 400 is electrically connected to the power 250 and the spectrometer 350 and configured to synchronize the power 250 and the spectrometer 350 so as to synchronize the production of the plasma 120 and the reception of the optical emission spectrum, achieve signal synchronization, synchronize the time period of receiving light signals by the light detection element 300 with the time period of producing the plasma 120 by the electrode 200, and effectively receive the light signals of the optical emission spectrum generated from the plasma 120.

In an embodiment, the electronic device 400 and the external device are smartphones, desktop computers, notebook computers, tablets, workstations, servers, cloud servers, and computing devices and provide user interfaces for users to operate. In a variant embodiment, the electronic device 400 and the external device are in telecommunication with another electronic device or another external device through which the electronic device 400 and the external device are indirectly operated or controlled. The electronic device 400, the external device, the power 250, and the spectrometer 350 further have an input module and an output module to provide visual and/or auditory user interfaces, such as display units, touchscreens, projectors, loudspeakers, phone voices, keyboards, mouses, dynamic detection, and speech recognition, for functioning as mediums of control and setting.

In an embodiment, the electrode is, for example, a glass platinum electrode and silver wire (CAS: 7440-22-4); the optical fiber functioning as the light detection element is, for example, FG600AEA manufactured by Thorlabs; the power is, for example, PSW 800-4.32 manufactured by GWINSTEK; the electronic device or circuit module is, for example, is Paspberry Pi 4 Model B/8 GB and Arduino Uno R3 and IGBT (insulated-gate bipolar transistor): individual or common combination of IXYP30N120C3; the spectrometer is, for example, 2030-025-FUV2A, Li-ion.

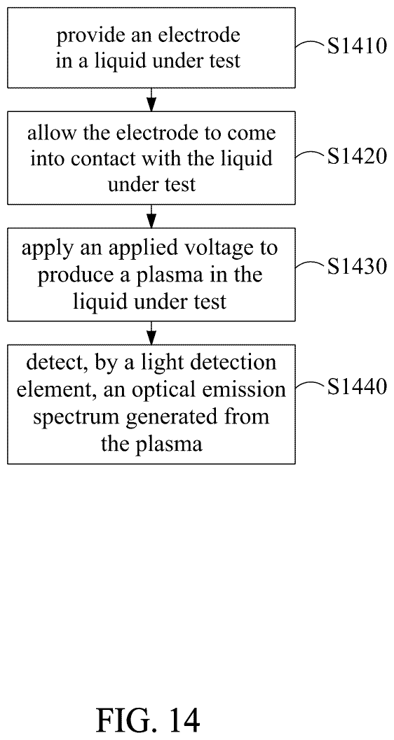

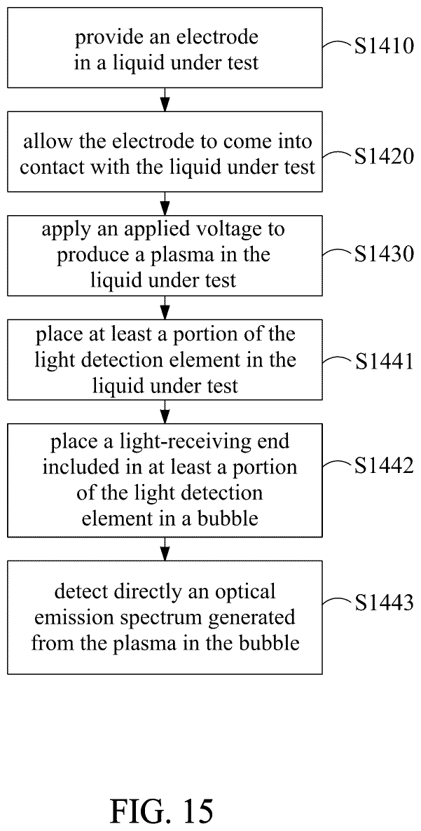

FIG. 14 is a schematic view of a process flow of a method of testing a substance in a liquid under test through plasma optical emission spectrum according to an embodiment of the disclosure. FIG. 15 is a schematic view of a process flow of the method of testing a substance in a liquid under test through plasma optical emission spectrum according to an embodiment of the disclosure.

FIG. 14 depicts the method of testing a substance in a liquid under test through plasma optical emission spectrum according to yet another aspect of the disclosure. The method is adapted to be carried out with the device or system of any one of the aforesaid embodiments of the disclosure, but the disclosure is not limited thereto. The method of testing a substance in a liquid under test through plasma optical emission spectrum according to an embodiment of the disclosure comprises the steps of: S1410 providing an electrode in a liquid under test; S1420 allowing the electrode to come into contact with the liquid under test; S1430 applying an applied voltage to produce a plasma in the liquid under test; and S1440 detecting, by a light detection element, an optical emission spectrum generated from the plasma. The plasma produced in S1430 is located in the bubble generated under the applied voltage. The light detection element in S1440 is an optical fiber, and no light-focusing component is present between the light detection element and the plasma.

Using an optical fiber not having any light-focusing component as a light detection element is effective in preventing plasma optical emission spectrum from undergoing signal attenuation and interference otherwise arising from unequal sizes of bubbles, changes and movements of bubbles, generation and destruction of bubbles, and optical phenomena, such as reflection and refraction caused by gas-liquid interfaces of bubbles, when the light signals are focused toward the optical fiber with optical components, such as lenses. Moreover, the light signals of the optical emission spectrum generated by plasma upon the generation of the plasma and bubbles in the liquid under test under an applied voltage are effectively and completely collected to obtain effective and precise results of analyses and tests of various substances in the liquid under test.

In an embodiment illustrated by FIG. 15, S1440 further comprises: S1441 placing at least a portion of the light detection element in the liquid under test; S1442 placing a light-receiving end included in at least a portion of the light detection element in a bubble; and S1443 detecting directly an optical emission spectrum generated from the plasma in the bubble.

The light-receiving end of the optical fiber not having any light-focusing component but functioning as a light detection element is directly placed in the liquid under test, and the light-receiving end functioning as the light detection element can be directly placed in a bubble including therein a plasma to minimize the effect of the bubble on light receiving, directly detect the optical emission spectrum generated from the plasma in the bubble, remain unaffected by interference from any bubbles or liquid, and greatly enhance the intensity and stability of the light signals of the plasma optical emission spectrum.

Therefore, the disclosure provides a device, system and method of testing a substance in a liquid under test through plasma optical emission spectrum, using an optical fiber not having any light-focusing component as a light detection element to effectively prevent plasma optical emission spectrum from undergoing signal attenuation and interference otherwise arising from marked interference-induced light receiving focus alteration because of unequal sizes of bubbles, changes and movements of bubbles, generation and destruction of bubbles, attachment of bubbles to the light detection element, and optical phenomena, such as reflection and refraction caused by gas-liquid interfaces of bubbles, when the light signals are focused toward the optical fiber with optical components, such as lenses. Owing to the optical fiber not having any light-focusing component, the light signals of the optical emission spectrum generated by plasma upon the generation of the plasma and bubbles in the liquid under test under an applied voltage are effectively and completely collected to obtain effective and precise results of analyses and tests of various substances in the liquid under test. The simple arrangement of the electrode and the light detection element is conducive to performing the tests through plasma optical emission spectrum, achieving advantages, such as portability, compactness, ease of use, and low cost, testing multiple heavy metals simultaneously, performing tests quickly, and rare mutual interference between different metals.

At least a portion of an optical fiber not having any light-focusing component but functioning as a light detection element is disposed within a sample region, and a light-receiving end of at least a portion of the light detection element is directly disposed within the scope of generation of bubbles, so as to directly detect optical emission spectrum generated from a plasma and located in a bubble and remain unaffected by any other bubbles and liquids. When the distance between the optical fiber and the electrode is short enough without affecting the generation of the plasma and the bubbles, resultant advantages are as follows: having the aforesaid advantages of the optical fiber; remaining unaffected by bubble-induced focus alterations, and shortening the distance between the light-receiving end and the plasma to reduce the interference of signal absorption or interfaces between the optical paths. Furthermore, when the light-receiving end of the optical fiber functioning as the light detection element is sufficiently close to the electrode and the plasma generated by the electrode, the light-receiving end of the optical fiber can be directly located in the bubble having therein the plasma to minimize the effect of the bubble on light receiving and greatly enhance the intensity and stability of the light signals of the plasma optical emission spectrum. With the optical fiber being sufficiently close to the bubble, the surface of the optical fiber is cleansed as a result of the generation, disappearance and alteration of the bubble outside the time period of detection of plasma light signals.

The integrated system and simple device of testing a substance in a liquid under test through plasma optical emission spectrum are applicable to different sites and scenes and can be conveniently carried to target waterbodies to perform a one-time test, or can be used to perform tests in a laboratory, or can be disposed at fixed addresses in any environments or plants to carry out real-time, persistent, long-term monitoring and provide test results, notices and alerts in real time without incurring high equipment cost and training cost or producing extra, undesirable by-products. Remote programmable control enables system information integration, automated controls, smart monitoring, and big data statistics.

The invention is disclosed above by preferred embodiments. However, persons skilled in the art should understand that the embodiments are illustrative of the invention only, but shall not be interpreted as restrictive of the scope of the invention. Please note that all variations and replacements equivalent to the embodiments shall be deemed falling within the scope of the invention, and the embodiments may be combined and changed in any ways. Therefore, the legal protection for the invention shall be defined by the appended claims.

While the present disclosure has been described by means of specific embodiments, numerous modifications and variations could be made thereto by those skilled in the art without departing from the scope and spirit of the present disclosure set forth in the claims.

What is claimed is:

1. A device of testing a substance in a liquid under test through plasma optical emission spectrum, comprising:
an electrode having at least a portion adapted to be disposed inside the liquid under test and adapted to be in contact with the liquid under test, wherein the electrode is adapted to produce a plasma in the liquid under test under an applied voltage, and the plasma is located in a bubble generated under the applied voltage; and
a light detection element adapted to detect an optical emission spectrum generated from the plasma in the bubble, the light detection element being an optical fiber, wherein no light-focusing component is present between the light detection element and the plasma.

2. The device of testing a substance in a liquid under test through plasma optical emission spectrum according to claim 1, wherein the light detection element has at least a portion adapted to be disposed inside the liquid under test and comprising a light-receiving end adapted to be located in the bubble to detect the optical emission spectrum generated from the plasma in the bubble.

3. The device of testing a substance in a liquid under test through plasma optical emission spectrum according to claim 1, wherein the light detection element is oriented at a first angle to a normal of a contact surface between the electrode and the liquid under test, with an included angle of 0° defined between the first angle and the normal of the contact surface.

4. The device of testing a substance in a liquid under test through plasma optical emission spectrum according to claim 3, wherein the light detection element has at least a portion adapted to be disposed inside the liquid under test and comprising a light-receiving end separated from the electrode by a distance configured to allow the light-receiving end to be adapted to be located within scope of the bubble.

5. The device of testing a substance in a liquid under test through plasma optical emission spectrum according to claim 3, wherein at least a portion of the light detection element comprises a light-receiving end separated from the electrode by a distance ranging from 0.1 mm to 4 mm.

6. The device of testing a substance in a liquid under test through plasma optical emission spectrum according to claim 1, wherein the light detection element is oriented at a second angle to a normal of a contact surface between the electrode and the liquid under test, with an included angle of 90° defined between the second angle and the normal of the contact surface.

7. The device of testing a substance in a liquid under test through plasma optical emission spectrum according to claim 6, wherein at least a portion of the light detection element is adapted to be disposed inside the liquid under test and comprises a light-receiving end separated from the electrode by a distance configured to allow the light-receiving end to be adapted to be located within scope of the bubble.

8. The device of testing a substance in a liquid under test through plasma optical emission spectrum according to claim 6, wherein at least a portion of the light detection element comprises a light-receiving end separated from the electrode by a distance ranging from 0.05 mm to 3.5 mm.

9. The device of testing a substance in a liquid under test through plasma optical emission spectrum according to claim 1, wherein the light detection element is oriented at a third angle to a normal of a contact surface between the electrode and the liquid under test, allowing an included angle defined between the third angle and the normal of the contact surface to fall between a first angle and a second angle, with an included angle of 0° defined between the first angle and the normal of the contact surface, with an included angle of 90° defined between the second angle and the normal of the contact surface.

10. The device of testing a substance in a liquid under test through plasma optical emission spectrum according to claim 9, wherein the light detection element has at least a portion adapted to be disposed inside the liquid under test and comprising a light-receiving end separated from the electrode by a distance configured to allow the light-receiving end to be adapted to be located within scope of the bubble.

11. The device of testing a substance in a liquid under test through plasma optical emission spectrum according to claim 9, wherein at least a portion of the light detection element comprises a light-receiving end separated from the electrode by a distance ranging from 0.05 mm to 3.5 mm.

12. The device of testing a substance in a liquid under test through plasma optical emission spectrum according to claim 1, wherein at least a portion of the light detection element is adapted to be disposed inside the liquid under test and comprises a light-receiving end separated from the electrode by a distance ranging from 0.05 mm to 10 mm.

13. A system of testing a substance in a liquid under test through plasma optical emission spectrum, comprising:
the device of claim 1;
a sample chamber configured to retain the electrode and the light detection element and receive the liquid under test;
a spectrometer coupled to the light detection element and configured to analyze an optical emission spectrum generated from a plasma in the bubble and detected by the light detection element; and
a power coupled to the electrode and configured to provide the applied voltage to the electrode.

14. The system of testing a substance in a liquid under test through plasma optical emission spectrum according to claim 13, further comprising an electronic device electrically connected to the spectrometer and configured to analyze the optical emission spectrum through the spectrometer.

15. The system of testing a substance in a liquid under test through plasma optical emission spectrum according to claim 14, wherein the electronic device is configured to be signal-connected to an external device to provide an analysis result about the optical emission spectrum of the liquid under test to the external device in real time.

16. The system of testing a substance in a liquid under test through plasma optical emission spectrum according to claim 13, further comprising an electronic device electrically connected to the power and configured to set a parameter of the applied voltage through the power to adjust the plasma produced in the liquid under test.

17. The system of testing a substance in a liquid under test through plasma optical emission spectrum according to claim 13, further comprising an electronic device electrically connected to the power and the spectrometer and configured to synchronize the power and the spectrometer so as to synchronize production of the plasma and reception of the optical emission spectrum.

18. A method of testing a substance in a liquid under test through plasma optical emission spectrum, comprising the steps of:
providing an electrode in a liquid under test;
allowing the electrode to come into contact with the liquid under test;
applying an applied voltage to produce a plasma in the liquid under test; and
detecting, by a light detection element, an optical emission spectrum generated from the plasma,
wherein the plasma is located in a bubble generated under the applied voltage,
wherein the light detection element is an optical fiber, and no any light-focusing component is present between the light detection element and the plasma.

19. The method of testing a substance in a liquid under test through plasma optical emission spectrum according to claim 18, wherein the step of detecting, by a light detection element, an optical emission spectrum generated from the plasma further comprises the steps of: placing at least a portion of the light detection element in the liquid under test; placing a light-receiving end included in at least a portion of the light detection element in a bubble; and detecting directly an optical emission spectrum generated from the plasma in the bubble.
